(19) United States
(12) Patent Application Publication
(10) Pub. No.: US 2025/0255512 A1
(43) Pub. Date: Aug. 14, 2025

(54) MONITORING METHOD AND RELATED PRODUCTS

(71) Applicant: Telesair, Inc., Irvine, CA (US)

(72) Inventors: Sreevalsan Sanathanan MENON, Santa Ana, CA (US); Yong LIU, Orange, CA (US); Stephen TUNNELL, Oceanside, CA (US)

(73) Assignee: Telesair, Inc., Irvine, CA (US)

(21) Appl. No.: 18/437,454

(22) Filed: Feb. 9, 2024

(51) Int. Cl. A61B 5/08; A61B 5/00; A61B 5/087

(52) U.S. Cl. CPC A61B 5/0816; A61B 5/0871; A61B 5/7257; A61B 2562/0247

(57) ABSTRACT

A monitoring method and related products. The method includes: obtaining sensing data measured by at least one sensing device, where the at least one sensing device is provided within the monitoring device and the sensing data characterizes a physical parameter of a gas flow at a side of the monitoring device, where the gas flow is delivered from the side of the monitoring device to a first user; and determining a first physiological parameter of the first user based on the sensing data.

### TECHNICAL FIELD

The present disclosure relates to the technical field of medical devices and, in particular, to a monitoring method and related products.

### BACKGROUND

In the treatment of respiratory diseases, monitoring a patient's physiological parameters is critical for optimizing the treatment of respiratory diseases. However, there are still many challenges to accurately monitor the physiological parameters.

This background information is provided to reveal information believed by the applicant to be of possible relevance to the present disclosure. No admission is necessarily intended, nor should be construed, that any of the preceding information constitutes prior art against the present disclosure.

### SUMMARY

The present disclosure provides a monitoring method and related products.

According to the method and related products, the monitoring of a physiological parameter of a first user (e.g., a patient with respiratory disease) can be conducted internally within a monitoring device itself, eliminating the necessity for measurements to be taken in close proximity to a patient circuit, improving the accuracy of monitoring the physiological parameters.

In a first aspect, an embodiment of the present disclosure provides a monitoring method, which is applied to a monitoring device and includes:
obtaining sensing data measured by at least one sensing device, where the at least one sensing device is provided within the monitoring device and the sensing data characterizes a physical parameter of a gas flow at a side of the monitoring device, where the gas flow is delivered from the side of the monitoring device to a first user; and
determining a first physiological parameter of the first user based on the sensing data.

In a second aspect, an embodiment of the present disclosure provides a monitoring device, including:

a memory stored with instructions and a processor, where the processor is configured to call and run the instructions stored in the memory to execute operations of:
obtaining sensing data measured by at least one sensing device, where the at least one sensing device is provided within the monitoring device and the sensing data characterizes a physical parameter of a gas flow at a side of the monitoring device, where the gas flow is delivered from the side of the monitoring device to a first user; and
determining a first physiological parameter of the first user based on the sensing data.

In a third aspect, an embodiment of the present disclosure provides a monitoring apparatus, and the monitoring apparatus includes:
an obtaining module, configured to obtain sensing data measured by at least one sensing device, where the at least one sensing device is provided within the monitoring apparatus and the sensing data characterizes a physical parameter of a gas flow at a side of the monitoring apparatus, where the gas flow is delivered from the side of the monitoring apparatus to a first user; and
a determining module, configured to determine a first physiological parameter of the first user based on the sensing data.

In a fourth aspect, an embodiment of the present disclosure provides a non-transitory computer-readable storage medium, which stores therein computer-executable instructions that, when being executed by one or more processors, implement the monitoring method according to the first aspect.

In a fifth aspect, an embodiment of the present disclosure provides a computer program, when the computer program is executed by one or more processors, implements the monitoring method according to the first aspect.

In a sixth aspect, an embodiment of the present disclosure provides a computer program product, which stores thereon computer-executable instructions which, when being executed by one or more processors, implements the monitoring method according to the first aspect.

It should be understood that the content described in this section is not intended to identify the key or important features of the embodiments of the present disclosure, nor to limit the scope of the present disclosure. Other features of the present disclosure will be easily understood through the following description.

### BRIEF DESCRIPTION OF THE DRAWINGS

The drawings are used for a better understanding of the present solution but do not constitute any limitation on the present disclosure. Throughout the drawings, identical or similar reference numbers designate identical or similar elements.

FIG. 1 is an exemplary schematic diagram of a monitoring system according to an embodiment of the present disclosure.

FIG. 2 is an exemplary schematic diagram of another monitoring system according to an embodiment of the present disclosure.

FIG. 3 is a schematic diagram of delta change that occurs for flow and pressure of a gas flow during breathing.

FIG. 4 is a schematic flowchart of a monitoring method according to an embodiment of the present disclosure.

FIG. 5 is a schematic diagram of a flow Fast Fourier transform (FFT) analysis for 17 breaths per minute of adult breathing.

FIG. 6 is a schematic diagram of a pressure FFT analysis for 17 breaths per minute of adult breathing.

FIG. 7 is a schematic diagram of a flow FFT analysis for set flows with 10 LPM to 60 LPM.

FIG. 8 is a schematic diagram of a pressure FFT analysis for set flows with 10 LPM to 60 LPM.

FIG. 9 is a schematic diagram of a window sliding of sliding starting from an initial position corresponding to an initial window.

FIG. 10 is a schematic diagram of flow rate of a gas flow over time during breath.

FIG. 11 is a schematic diagram of pressure of a gas flow over time during breath.

FIG. 12 is a schematic diagram of a flow rate and pressure peak analysis of a gas flow during breath.

FIG. 13 is a schematic structural diagram of a monitoring apparatus according to an embodiment of the present disclosure.

FIG. 14 is a schematic structural diagram of a monitoring device according to an embodiment of the present disclosure.

### DETAILED DESCRIPTION OF THE EMBODIMENTS

In the following description, reference is made to the accompanying figures, which form part of the disclosure, and which show, by way of illustration, specific aspects of embodiments of the present disclosure or specific aspects in which embodiments of the present disclosure may be used. It is understood that embodiments of the present disclosure may be used in other aspects and include structural or logical changes not depicted in the figures. The following detailed description, therefore, is not to be taken in a limiting sense, and the scope of the present disclosure is defined by the appended claims.

The term “include” used herein and its variations are open inclusion, that is, “include but not limited to”. The term “based on” means “at least partly based on”. The term “an embodiment” represents “at least one embodiment”; the term “another embodiment” represents “at least one another embodiment”; and the term “some embodiments” represents “at least some embodiments”. Related definitions of other terms will be provided in the following.

It should be noted that concepts such as “first”, and “second” mentioned in the present disclosure are merely used to distinguish different apparatuses, modules, or units, but not to limit the sequence or interdependency of functions executed by these apparatuses, modules, or units.

It should be noted that the singular or plural modification mentioned in the present disclosure is illustrative and not restrictive, and those skilled in the art should understand that it should be understood as “one or more” unless clearly defined in the context otherwise.

In the treatment of respiratory diseases, a patient's physiological parameters, especially related to breathing such as breath rate, breath cycle, and other physiological parameters are typically monitored by an interface of a sensor set near the patient. However, in this way, the measurement of these parameters is challenging. For example, high-flow systems provide variable and often turbulent airflow, making it more complex to accurately measure the patient's physiological parameters related to breathing compared to traditional ventilation methods. High-flow systems typically do not involve a direct connection to the patient's airway, unlike invasive ventilation methods. This lack of direct interaction makes breath rate monitoring less straightforward. Patients using high-flow nasal cannulas may experience displacement or removal of the cannula during coughing or movement, potentially interrupting breath rate measurements. The leak flow and use of different nasal cannulas also affect the accuracy of breath rate measurements.

In view of the above problem, the present disclosure provides a monitoring method and related products. According to the method and related products, the monitoring (measuring) of a physiological parameter of a first user (e.g., a patient with respiratory disease) can be conducted internally within a monitoring device itself, eliminating the necessity for measurements to be taken in close proximity to a patient circuit, thereby solving the problem of the inaccuracy measurement of the physiological parameter due to t, as discussed above.

It should be noted that the above description is only intended to illustrate the technical conception of the present disclosure by taking specific examples and is not intended to limit the application scenarios of the present disclosure to the monitoring (measuring) of the physiological parameters (e.g., breathing rate, breathing cycle, and so on) related to breathing in high-flow systems, that is, the solution proposed by the present disclosure can be applied to the monitoring (measuring) of physiological parameters that are related to breathing or other than the physiological parameters related to breathing in high-flow systems or other respiratory treatment systems (e.g., noninvasive ventilation systems, invasive ventilation systems, or the like).

Before elaborating on the embodiments of the present disclosure, an exemplary scenario to which the embodiments are applicable will be described in the first place.

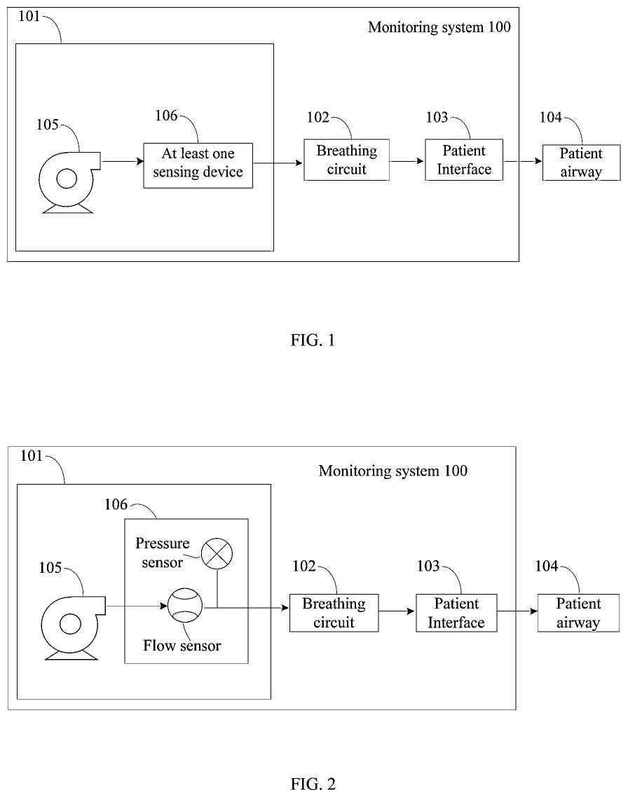

FIG. 1 is an exemplary schematic diagram of a monitoring system according to an embodiment of the present disclosure. With reference to FIG. 1, a monitoring system 100 includes a monitoring device 101, a breathing circuit 102, and a patient interface 103.

The monitoring device 101 can include a flow source 105 and at least one sensing device 106. The monitoring device 101 is configured to monitor a first physiological parameter of the first user with a respiratory disease. Here the first physiological parameter may reflect the health condition of the first user, it could be, e.g., breathing rate, breathing cycle, or the like. In an implementation, the monitoring device 101 can be a high-flow device for providing a high-flow rate of oxygen to patients and monitoring physiological parameters of patients, or other respiratory therapy devices for providing ventilation support and monitoring physiological parameters of patients. In an implementation, the monitoring device 101 can be integrated into a respiratory therapy device to monitor the first physiological parameter of patients.

The flow source 105 can deliver a gas flow to the first user. The gas flow used herein may refer to a flow of oxygen, air, or oxygen-containing gas. Oxygen refers to pure oxygen or high-concentration oxygen (95% or above) that is used in oxygen therapy. The oxygen-containing gas refers to a mixture of oxygen and air for delivery to a patient. The proportion of oxygen in the oxygen-containing gas is not specifically limited in the present disclosure. In an implementation, the gas flow can be provided by the monitoring device, or provided by a respiratory therapy device integrated with the monitoring device. The flow source 105 may be a blower that can deliver the gas flow, or a turbine that can run oxygen and air mixture to deliver the gas flow, and the like.

In an implementation, the monitoring device 101 may include the flow source 105 (e.g., the monitoring device 101 can be a high-flow device, in this case the monitoring device 101 can also be referred to as a respiratory therapy device), for example, the monitoring device 101 can deliver the gas flow from the flow source 105 to the first user (i.e., the gas flow is delivered from the side of the monitoring device to the first user), and detect the gas flow to the first user. In an implementation, the monitoring device 101 may not include the flow source 105, the monitoring device 101 is integrated into the respiratory therapy device, the respiratory therapy device equipped with the flow source 105 provides and delivers the gas flow to the first user, and the monitoring device 101 can detect the gas flow to the first user (i.e., the gas flow can be delivered from the side of the monitoring device to the first user).

The at least one sensing device 106 provided within the monitoring device 101 can be configured to obtain sensing data. The sensing data characterizes a physical parameter of the gas flow at a side of the monitoring device 101, where the gas flow is delivered from the side of monitoring device 101 (i.e., delivered from the flow source mentioned above) to the first user, that is, the at least one sensing device can be configured to measure the physical parameter of the gas flow. Herein the number of the at least one sensing device is not limited in the present disclosure.

In an implementation, the at least one sensing device 106 may be at least one flow sensor, at least one pressure sensor, or a combination of at least one flow sensor and at least one pressure sensor. Correspondingly, the physical parameter may include a flow rate of the gas flow at the side of the monitoring device sensed by the at least one flow sensor, a pressure of the gas flow at the side of the monitoring device sensed by the at least one pressure sensor, or the flow rate of the gas flow and the pressure of the gas flow at the side of the monitoring device sensed by the combination of the at least one flow sensor and at least one pressure sensor. The at least one flow sensor is used for feedback a signal to control the gas flow to a set value in the monitoring device. The at least one pressure sensor is used to detect the pressure inside the monitoring device when a certain gas flow is delivered, depending upon the cannula, patient, leak, and flow setting the pressure reading can vary. In a case the at least one sensing device 106 includes a pressure sensor and a flow sensor, the flow sensor and the pressure sensor can be provided between the flow source 105 and the breathing circuit 102, as shown in FIG. 2.

The breathing circuit 102 allows delivery of the gas flow to the patient interface 103 (e.g., a nasal cannula, or the like), and the patient interface 103 is used to connect to a patient airway 104 of the first user.

The inventor of the present disclosure found by the study that since the monitoring system 100 is connected to the first user (patient airway 104 of the first user), as a physiological condition (e.g., breathing pattern) of the first user changes, the physical parameter of the gas flow at the side of the monitoring device 101 may change. For example, the respiratory therapy device (e.g., a high-flow device) generates a dynamic positive pressure impact through the interaction of high inspiratory flow and the patient interface, and this results in the creation of a noticeable yet subtle positive pressure. This variation in the dynamic positive pressure gets influenced by the patient's breathing patterns and can be effectively harnessed to estimate the first physiological parameter of the first user, for example, a physiological parameter (e.g., breathing rate, breathing cycle, or the like) related to breathing. Likewise, the delivered gas flow experiences fluctuations as the patient inhales and exhales, and monitoring these variations in delivered gas flow provides another valuable means of detecting the first physiological parameter of the first user.

In case the monitoring device is integrated into the respiratory therapy device (e.g., the high-flow device), the monitoring device can be configured to monitor the variation in the dynamic positive pressure and/or the variation in delivered gas flow, so as to detect the first physiological of the first user. In case the monitoring device is the respiratory therapy device (e.g., the high-flow device), the monitoring device can generate the dynamic positive pressure impact and provide the delivered gas flow, and can be configured to monitor the variation in the dynamic positive pressure and/or the variation in delivered gas flow, so as to detect the first physiological of the first user.

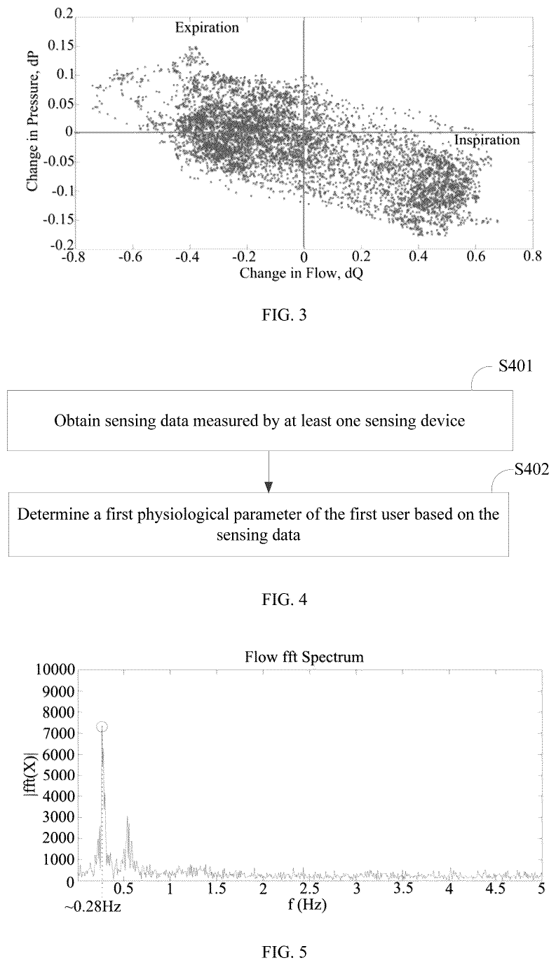

FIG. 3 is a schematic diagram of delta change that occurs for flow and pressure of a gas flow during breathing. As shown in FIG. 3, data in a horizontal axis (the x-axis in a traditional Cartesian coordinate system) direction shows the delta change in flow and it increases during inspiration and decreases during expiration; and data in a vertical axis (the y-axis in the traditional Cartesian coordinate system) direction shows the delta change in pressure and it drops during inspiration and increases during exhalation. By utilizing both pressure and flow as indicators, the first physiological parameter (e.g., physiological parameters related to breathing) of the first user can be estimated.

Therefore, based on the sensing data, from the at least one sensing device provided within the monitoring device, that characterizes the physical parameter of the gas flow at the side of the monitoring device, where the gas flow is delivered from the side of the monitoring device to the first user, the first physiological parameter of the first user can be determined, thereby the monitoring of the first physiological parameter of the first user can be realized internally within the monitoring device itself, eliminating the necessity for measurements to be taken in close proximity to a patient circuit, and improving the accuracy of monitoring the physiological parameters.

It should be understood that the monitoring system 100 can include other universal devices such as one or more processors, one or more memories, one or more communication interfaces, and the like. The one or more memories can be configured to store a computer program/computer instructions. The one or more processors can be configured to execute the computer program/computer instructions to control the operations of the respective devices/apparatuses in the monitoring system via a communication connection, such as a direct wired or wireless connection, or via any kind of network, e.g. a wired or wireless network or any combination thereof, or any kind of private and public network, or any kind of combination thereof. The monitoring device can exchange data with other devices by the one or more communication interfaces.

It is understood that FIG. 1 is merely a logical schematic diagram of the monitoring system 100, which shows an exemplary configuration of functional units/modules. In practical application scenarios, the function units of the system may be implemented in various forms, which are not limited by the embodiments of the present disclosure.

FIG. 4 is a schematic flowchart of a monitoring method according to an embodiment of the present disclosure. The monitoring method can be applied to the monitoring system 100 mentioned above, and more specifically, applied to the monitoring device 101. Specifically, the monitoring method includes the steps as follows.

Step S401, the monitoring device obtains sensing data measured by at least one sensing device.

The monitoring device is configured to monitor the physiological condition of a first user (e.g., by monitoring physiological parameters) who uses the monitoring device to monitor the physiological condition himself/herself or whose physiological condition is monitored by a second user operating the monitoring device (e.g., a physician, or relatives and or friends of the first user). The first user may be a patient with respiratory disease or other users whose physiological parameters need to be monitored. In an implementation, the monitoring device can be a high-flow device for providing a high-flow rate of oxygen to patients, or other respiratory therapy devices for providing ventilation support. In an implementation, the monitoring device can be integrated into respiratory therapy devices (e.g., the high-flow device, noninvasive ventilation device, invasive ventilation device, or the like) for providing ventilation support.

The at least one sensing device is provided within the monitoring device and the sensing data characterizes a physical parameter of a gas flow at a side of the monitoring device, and the gas flow is delivered from the side of the monitoring device to the first user. As discussed in the description of FIG. 1, the gas flow used herein may refer to a flow of oxygen, air, or oxygen-containing gas provided to the first user for ventilation support of the first user. In an implementation, the at least one sensing device can include at least one flow sensor, and the physical parameter includes a flow rate of the gas flow at the side of the monitoring device. In an implementation, the at least one sensing device can include at least one pressure sensor, and the physical parameter includes the pressure of the gas flow at the side of the monitoring device. In an implementation, the at least one sensing device can include at least one flow sensor and at least one pressure sensor, and the physical parameter includes a flow rate of the gas flow and the pressure of the gas flow at the side of the monitoring device.

Step S402, the monitoring device determines a first physiological parameter of the first user based on the sensing data.

In an implementation, the sensing data based on which the monitoring device can determine the first physiological parameter of the first user can be directly obtained from the at least one sensing device, or obtained by performing preprocessing on the sensing data directly from the at least one sensing device. In an implementation, the preprocessing may include at least one of the following: data set selection, data filtering, data cleaning, and other data processing manners that are well-known in the art and are not described in detail herein.

In an implementation, the first physiological parameter can be a physiological parameter related to breathing (breathing rate, breathing cycle, or the like), or other physiological parameters, which is not limited in the present disclosure.

As illustrated in connection with FIG. 1, as the physiological condition of the first user changes, the physical parameter of the gas flow at the side of the monitoring device may change. Therefore, according to the monitoring method, based on the sensing data measured by the at least one sensing device provided within the monitoring device, where the sensing data characterizes the physical parameter of the gas flow at the side of the monitoring device, and the gas flow is delivered from the side of the monitoring device to the first user, the first physiological parameter of the first user can be determined. Since the measurement process of the physical parameter and analysis is conducted internally within the monitoring device itself, an external monitoring device (e.g., a sensor provided in close proximity to a patient circuit) is no longer needed, solving the problem of the inaccuracy measurement of the first physiological parameter due to e.g., the variable flow rates, the absence of a direct connection, or potential sensor interface (e.g., cannula) displacement in the traditional measurement manner (i.e., measuring through the sensor provided in close proximity to the patient circuit), thereby improving the accuracy of monitoring physiological parameters.

In an implementation, step S402 of determining the first physiological parameter of the first user based on the sensing data can include: the monitoring device determines the first physiological parameter of the first user based on peaks of the sensing data or changing rates derived from the sensing data in a time domain or a maximum frequency component of the sensing data in a frequency domain.

In this implementation, after obtaining the sensing data, the monitoring device can process the sensing data in the time domain or the frequency domain. In an implementation, the peaks refer to peaks of the sensing data over a predefined time length. In an implementation, the changing rates refer to a plurality of fit lines obtained by performing fitting on the sensing data. In an implementation, the maximum frequency component refers to a frequency component with the maximum amplitude in the frequency domain, and a frequency corresponding to the maximum frequency component represents a signal that is most repetitive in the sensing data. Since the pattern of the first physiological parameter is typically regular, all of the peaks of the sensing data, the changing rates, and the maximum frequency component can be used to determine a changing pattern of the first physiological parameter, and then derive the first physiological parameter.

In the following, a detailed description will be made with respect to how to determine the first physiological parameter based on the peaks of the sensing data or the changing rates derived from the sensing data, in the time domain or the maximum frequency component of the sensing data in the frequency domain by taking an example where the first physiological parameter relates to breathing. It should be understood by those skilled in the art that the examples are only intended to illustrate the technical conception, but not intended to limit the first physiological parameter to physiological parameters related to breathing, that is, it may be other physiological parameters.

In an implementation, determining the first physiological parameter of the first user based on the maximum frequency component of the sensing data in the frequency domain can include the following steps:
step S501, the monitoring device applies Fourier transform to the sensing data to obtain the maximum frequency component of the sensing data; and
step S502, the monitoring device determines a frequency corresponding to the maximum frequency component as the first physiological parameter.

In this implementation, the monitoring device applies a Fourier transform to the continuously monitored flow rate data (i.e., the sensing data) over a specified time range sampled at a specified frequency (i.e., specified sampling frequency) to obtain the maximum frequency component of the sensing data. The Fourier transform can be performed using standard Discrete Fourier Transform (DFT) by the following formula:

$$Y ( k ) = {\sum }_{j = 1}^{n} X ( j ) {W}_{n}^{( j - 1 ) ( k - 1 )} ;$$

where Y(k)=ƒƒt(X) that is, a result of the Fast Fourier Transform of X, X represents flow rate data (i.e., the sensing data characterizing the flow rate and/or pressure of the gas flow at the side of the monitoring device); Wₙ=e^{(-2 πi)/n}, i is an imaginary unit, n represents the length of the calculated sequence, that is, the number of sampling points; j is a positive integer from 1 to n; k represents frequency.

The specified time range is a time range over which the flow rate data (i.e., the sensing data) is continuously monitored. The specified sampling frequency refers to the number of times a signal is sampled per second. In this implementation, the specified time range can be not less than 1 minute, for example, 1 to 60 minutes, or other time ranges. The selection of the specified sampling frequency relates to the Nyquist-Shannon sampling theorem. The specified sampling frequency can be 10 times or more whatever is the lowest, or highest frequency that is going to be measured. For example, if the frequency of interest is 3 Hz, the specified sampling frequency can be 30 Hz or more, e.g., 50 Hz.

This frequency analysis enables to identify the frequency at which flow rate variations occur. The maximum frequency component of the sensing data, that is, the frequency component with the highest magnitude can then be extracted to determine the first physiological parameter. Specifically, the frequency corresponding to the maximum frequency component can be determined as the first physiological parameter. The first physiological parameter may be breathing rate in this implementation. Accordingly, the breathing cycle can also be determined based on the breathing rate, that is, the reciprocal of the breathing rate.

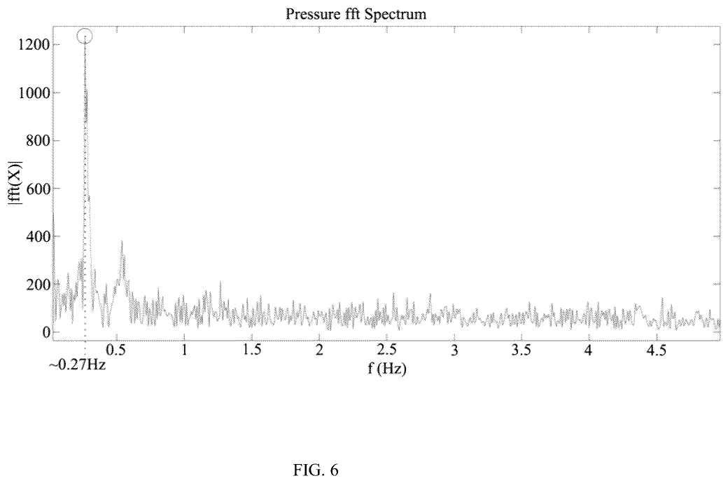

In this implementation, both the flow rate and the pressure of the gas flow can be used to determine the first physiological parameter based on the frequency analysis, as shown in FIG. 5 and FIG. 6 respectively.

FIG. 5 is a schematic diagram of a flow Fast Fourier transform (FFT) analysis for 17 breaths per minute of adult breathing, in which the x-axis represents frequency, the y-axis represents a result (the absolute value of FFT) of applying FFT to the continuously monitored flow rate of the gas flow at the side of the monitoring device over 1 minute sampled at 50 Hz. The frequency corresponding to the peak represents the signal that is most repetitive in the data. The dominant signal frequency (maximum frequency component) will be that of the breath and finding the frequency with the highest magnitude corresponds to the breath rate. Correspondingly, as shown in FIG. 5, the frequency with the highest magnitude (i.e., the frequency corresponding to the maximum frequency component) is about 0.28 Hz, that is, the breathing rate extracted from FIG. 5 is 0.28 Hz (i.e., 0.28×60˜17 breaths per minute), and a breathing cycle is about 4 seconds (I/O.28≈4).

FIG. 6 is a schematic diagram of a pressure FFT analysis for 17 breaths per minute of adult breathing, in which the x-axis represents frequency, the y-axis represents a result (the absolute value of FFT) of applying FFT to the continuously monitored pressure of the gas flow at the side of the monitoring device over 1 minute sampled at 50 Hz. As shown in FIG. 6, the frequency with the highest magnitude (i.e., the frequency corresponding to the maximum frequency component) is about 0.27 Hz, that is, the breathing rate extracted from FIG. 5 is 0.27 Hz (i.e., 0.27×60˜17 breaths per minute), and a breathing cycle is about 4 seconds (I/O.27≈4).

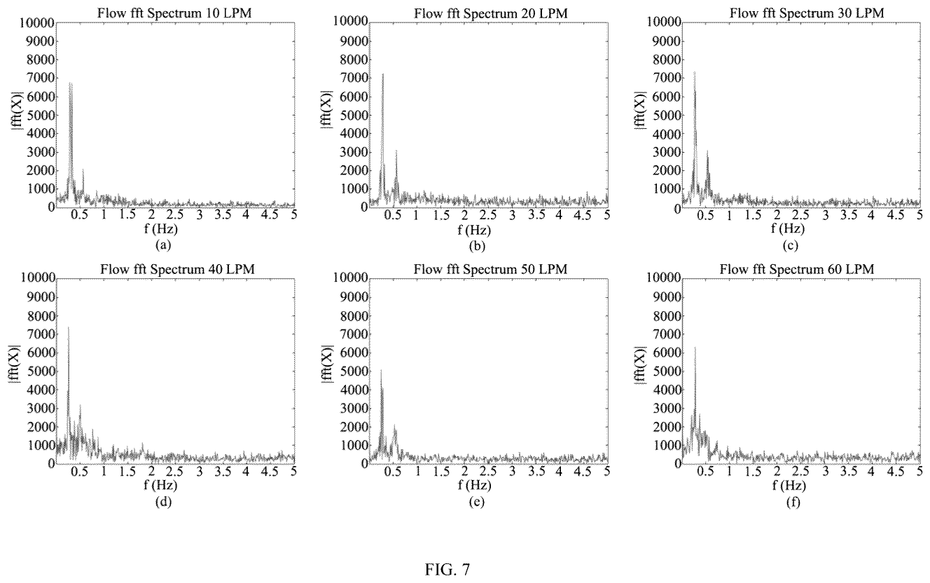

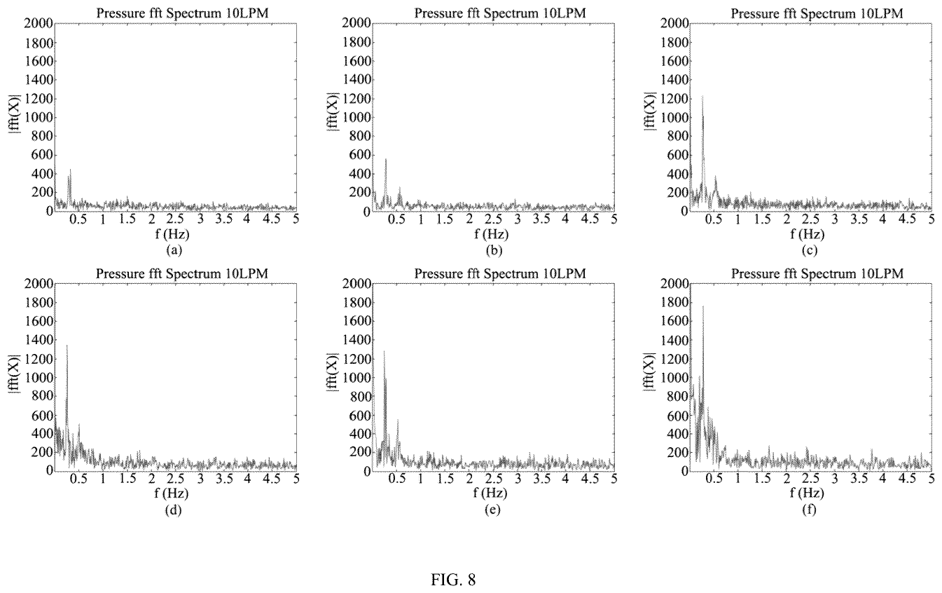

In order to prove the method mentioned above can successfully find the breathing rate for different set flows, the present disclosure further provides FIG. 7 and FIG. 8, in which figures (a) to (f) show flow FFT analysis and pressure FFT analysis for set flows with 10 LPM to 60 LPM respectively. As shown in FIG. 7 and FIG. 8, an obvious maximum frequency component can be found in the drawings to realize deriving the breathing rate in a simple way, and this shows independent of set flow the present disclosure can utilize the Fourier transform method.

In this implementation, the determination of the first physiological parameter of the first user based on the maximum frequency component of the sensing data in the frequency domain can be enhanced through windowed analysis (also called sliding window analysis), allowing for real-time updates of the breathing rate. For windowed analysis, a window of the signal data (i.e., the sensing data) is extracted and FFT is performed on that window. The large vectors of data (i.e., the sensing data after being performed FFT) can be captured and a small window size can be used through the data to find the frequency with the highest magnitude. This window can be slid through the data to find the frequency from a different signal segment. In this way, multiple/same values of the frequency can be obtained and they can be averaged to find the breathing rate (i.e., averaged breathing rate).

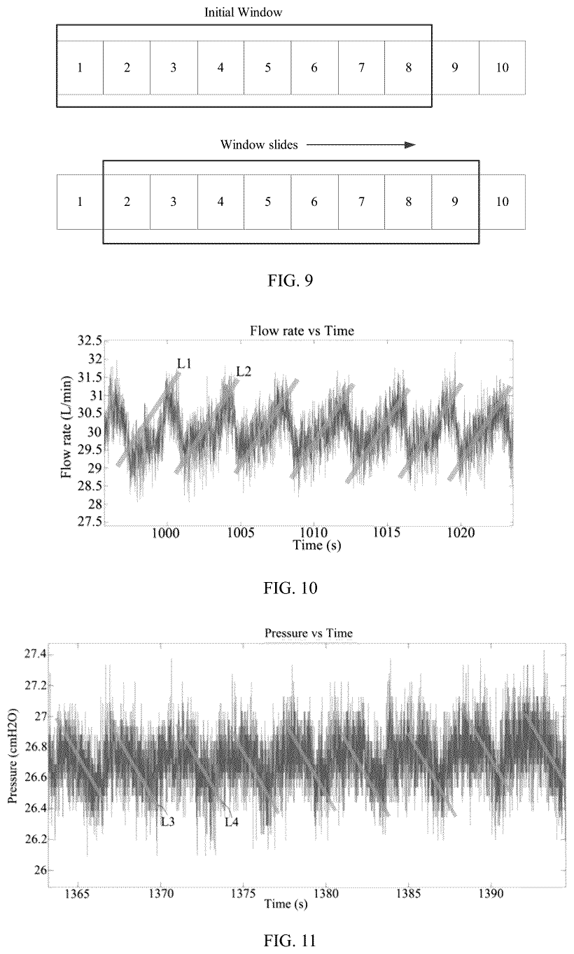

In the window analysis method, a window size and sliding step size for the windowed analysis can be defined. FIG. 9 is a schematic diagram of a window sliding of sliding starting from an initial position corresponding to an initial window. As shown in FIG. 9, the window size can be 8 seconds, and the sliding step size can be 1 second. For example, if the window size (length) is 15 seconds, the sliding window method can detect at least 4 BPM (Breaths Per Minute).

In an implementation, determining the first physiological parameter of the first user based on the changing rates derived from the sensing data in the time domain can include the following steps:
step S601, the monitoring device performs fitting on the sensing data to obtain a plurality of fit lines, where changing rates of the plurality of fit lines are all negative or positive;
step S602, the monitoring device calculates a time interval between at least one pair of fit lines among the plurality of fit lines; and
step S603, the monitoring device determines the first physiological parameter according to the time interval.

The solution mentioned above is described below in detail in connection with FIG. 10 and FIG. 11.

FIG. 10 is a schematic diagram of a flow rate of a gas flow over time during breathing, in which the horizontal axis represents time, in the unit of second(s); the vertical axis represents the flow rate of the gas flow at the side of the monitoring device, in L/min (liter/minute).

By adopting the solution, it is possible to determine at least the breathing rate or breathing cycle by tracking changing rates of the flow rate curve. In step S601, since the sensing data is normally analogue data, so the monitoring device can first perform fitting on the sensing data (i.e., the flow rate of the gas flow over time in this example described in connection with FIG. 10) to obtain a plurality of fit lines (e.g., oblique thick lines such as those labeled with L1 and L2), and then changing rates of the plurality of fit lines can be obtained. It can be seen that the changing rates of the plurality of fit lines are all positive in this example. A noticeable increase in the changing rates corresponds to an inhalation, making this rise in flow rate a key indicator for detecting breaths and estimating the breathing rate. The calculation of the changing rate can be performed using linear regression to find the changing rates of the detected signal (the flow rate of the gas flow).

In step S602, the monitoring device can calculate a time interval between at least one pair of fit lines among the plurality of fit lines. Since breathing is typically regular, the changing rates of the plurality of fit lines are almost the same, a time interval between one pair of adjacent fit lines among the plurality of fit lines is one breathing cycle, and a time interval between at least two adjacent fit lines or at least one pair of non-adjacent fit lines among the plurality of fit lines is an integer (the integer is greater than or equal to 2) multiple of one breathing cycle. Therefore, the first physiological parameter (breathing rate in this example) can be determined according to the time interval in step S603.

In the case the time interval in step S602 is a first time interval between one pair of adjacent fit lines among the plurality of fit lines, the first physiological parameter (breathing rate) is determined as the reciprocal of the first time interval.

In the case the time interval in step S602 is a second time interval between at least two pairs of adjacent fit lines among the plurality of fit lines, the first physiological parameter (breathing rate) is determined as the reciprocal of a first averaged time interval. The first averaged time interval is equal to the second time interval divided by the number of pairs of adjacent fit lines.

In the case the time interval in step S602 is a third time interval between at least one pair of non-adjacent fit lines among the plurality of fit lines, the first physiological parameter (breathing rate) is determined as the reciprocal of a second averaged time interval. The third time interval is the sum of time intervals (i.e., one breathing cycle) between each pair of the non-adjacent fit lines, for example, for two pairs of non-adjacent fit lines (L1 and L3, L5 and L7), there are two time intervals between L1 and L3, there are two time intervals between L5 and L7, and the sum of time intervals is the duration of four time intervals (i.e., four breathing cycles). In an implementation, the second averaged time interval is equal to the third time interval divided by the sum of the number of the time intervals between each pair of the non-adjacent fit lines, for example, for the two pairs of non-adjacent fit lines (L1 and L3, L5 and L7), the sum of the number of the time intervals between each pair of the non-adjacent fit lines is four, and the second averaged time interval is equal to the duration of four time intervals divided by four.

For example, in the case the time interval in step S602 is the second time interval, if the averaged time interval is calculated for three pairs of adjacent fit lines (e.g., lines L1 to L4), the averaged time interval is equal to the second time interval divided by the number (i.e., 3) of pairs of adjacent fit lines. For another example, in the case the time interval in step S602 is the third time interval, if the averaged time interval is calculated for one pair of non-adjacent fit lines (e.g., lines L1 and L4), the averaged time interval is equal to the third time interval divided by the difference (i.e., 3) between the number (i.e., 4) of fit lines among one pair of non-adjacent fit lines and the number (i.e., 1) of pairs of the non-adjacent fit lines; in the case the time interval in step S602 is the third time interval, if the averaged time interval is calculated for two pairs of non-adjacent fit lines (e.g., lines L1 and L3, and lines L5 and L7), the averaged time interval is equal to the third time interval divided by the difference (i.e., 4) between the number (that is, the number (i.e., 6) of lines L1, L2, L3, L5, L6 and L7) of fit lines among the two pairs of the non-adjacent fit lines and the number (i.e., 2) of pairs of the non-adjacent fit lines.

In an implementation, the time interval can be obtained by using a timer. For example, in response to detecting a change (i.e., the change refers to the changing of the changing rate from positive to negative or from negative to positive) of the changing rate of interest, a timer can be started at a first time until detecting a next change of the changing rate at a second time, and the time interval between the first time and the second time is the breathing cycle.

The solution previously explained for flow rate analysis can also be extended to pressure monitoring for breathing rate detection. When a patient inhales, the pressure of the gas flow at the side of the monitoring device initially decreases and subsequently rises, which corresponds to and is contrary to the changing pattern of the flow rate of the gas flow at the side of the monitoring device. This distinctive changing rate pattern of pressure can also serve as a reliable indicator for identifying the breath cycle.

FIG. 11 is a schematic diagram of pressure of a gas flow over time during breathing, in which the horizontal axis represents time, in seconds, and the vertical axis represents the pressure of the flow gas at the side of the monitoring device, in cmH2O. As shown in FIG. 11, a plurality of fit lines (e.g., oblique thick lines such as those labeled with L3 and L4) can be also obtained by fitting on the sensing data (i.e., the pressure of the gas flow over time in this example described in connection with FIG. 11), and then changing rates of the plurality of fit lines can be obtained. It can be seen that the changing rates of the plurality of fit lines are all positive in this example. Next, a time interval between at least one pair of adjacent or non-adjacent fit lines among the plurality of fit lines can be calculated to determine the first physiological parameter according to the time interval.

In the case the time interval in step S602 is a first time interval between one pair of adjacent fit lines among the plurality of fit lines, the first physiological parameter (breathing rate) is determined as the reciprocal of the first time interval.

In the case the time interval in step S602 is a second time interval between at least two pairs of adjacent fit lines among the plurality of fit lines, the first physiological parameter (breathing rate) is determined as the reciprocal of a first averaged time interval. The first averaged time interval is equal to the second time interval divided by the number of pairs of adjacent fit lines.

In the case the time interval in step S602 is a third time interval between at least one pair of non-adjacent fit lines among the plurality of fit lines, the first physiological parameter (breathing rate) is determined as the reciprocal of a second averaged time interval. The third time interval is the sum of time intervals (i.e., one breathing cycle) between each pair of the non-adjacent fit lines, for example, for two pairs of non-adjacent fit lines (L1 and L3, L5 and L7), there are two time intervals between L1 and L3, there are two time intervals between L5 and L7, and the sum of time intervals is the duration of four time intervals (i.e., four breathing cycles). In an implementation, the second averaged time interval is equal to the third time interval divided by the sum of the number of the time intervals between each pair of the non-adjacent fit lines, for example, for the two pairs of non-adjacent fit lines (L1 and L3, L5 and L7), the sum of the number of the the time intervals between each pair of the non-adjacent fit lines is four, and the second averaged time interval is equal to the duration of four time intervals divided by four.

In an implementation, determining the first physiological parameter of the first user based on the peaks of the sensing data in the time domain can include the following steps: step S701, the monitoring device calculates a number of peaks of the sensing data over a predefined time length; and step S702, the monitoring device obtains the first physiological parameter based on the number of peaks and the predefined time length.

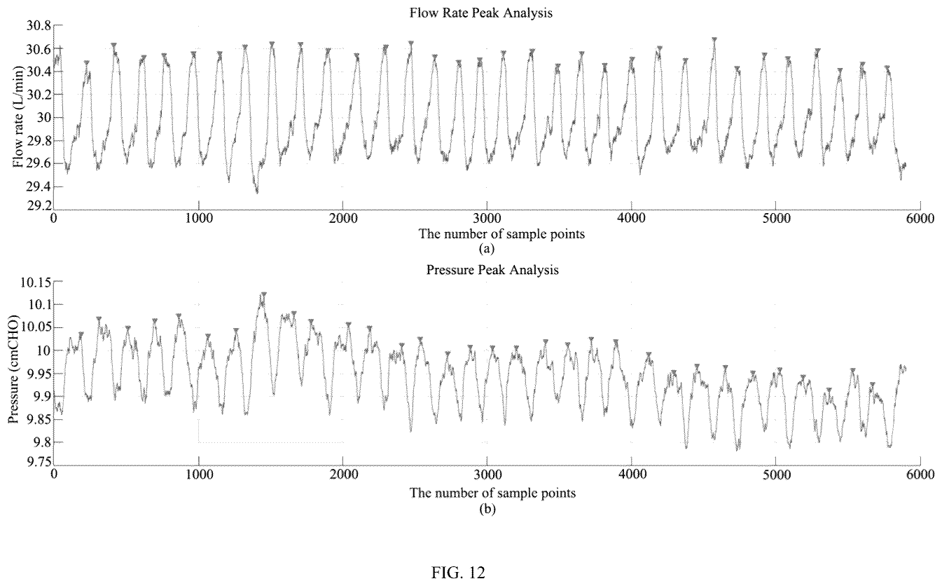

FIG. 12 is a schematic diagram of a flow rate and pressure peak analysis of a gas flow during breathing, in which the both horizontal axes represent the number of sampling points over time, the figure (a) of the vertical axis represents the flow rate of the gas flow at the side of the monitoring device over time, and the figure (b) of the vertical axis represents the pressure of the gas flow at the side of the monitoring device over time. In step S701, the monitoring device can calculate the number of peaks of the sensing data (i.e., the flow rate or the pressure in this implementation) over a predefined time length, where the peaks of the flow rate are labelled with inverted triangles in figure (a) of FIG. 12, and the peaks of the pressure are labelled with inverted triangles in figure (b) of FIG. 12. In an implementation, the predefined time length may be at least 1 minute or other time range more than 1 minute.

In step S702, the monitoring device can obtain the first physiological parameter based on the number of peaks and the predefined time length. For example, if peak analysis shows 4 peaks in 15 seconds, then the breathing rate is 16 breaths per minute since there are four 15 seconds in per minute.

In the implementations mentioned above, both the flow rate and the pressure of the gas flow at the side of the monitoring device can be used to determine the breathing rate. To enhance measurement precision, a combined approach can be adopted by integrating both the flow rate and pressure analysis solutions mentioned above. Specifically, both the signal data (the flow rate and pressure signal data) can be used to confirm whether it is a real breath. For example, if the flow rate analysis shows it's a real breath and the pressure analysis shows it's not a real breath, then the case is not considered as a real breath. Only if both analyses show it's a real breath it can be considered as a real breath.

The respiratory pattern as shown in FIG. 3 can be used to confirm whether it is a real breath, for example, it can determine whether the pressure analysis of the gas flow meets the respiratory pattern in which the pressure of the flow gas drops during the inspiration of a breath and increases during the expiration of the breath. If so, it can be determined that both the pressure and flow rate analyses of the gas flow show it is a real breath, and it can be considered as a real breath; if not, it can be determined it cannot be considered as a real breath. For another example, a breathing rate derived by the flow rate analysis solutions can be compared with a breathing rate derived by the pressure analysis solutions, if they are the same, the derived breathing rate can be considered as an accurate breathing rate.

In an implementation, in case the physical parameter is the flow rate of the gas flow, step S601 can include: for the plurality of fit lines, determining whether changing rates of at least one pair of fit lines among the plurality of fit lines are all positive; if the changing rates of the at least one pair of fit lines among the plurality of fit lines are all positive, it is determined that breath(s) corresponding to the at least one pair of fit lines is real breath(s), and then the monitoring device can continue the execution of steps S602 and S603 based on the sensing data (the sensing data corresponding to the at least one pair of fit lines); if the changing rates of the at least one pair of fit lines among the plurality of fit lines are not all positive (i.e., at least one changing rate of at least one fit line is negative), it is determined that a breath corresponding to the fit line(s) with a negative changing rate is not a real breath, sensing data corresponding to the fit line(s) with a negative changing rate is deleted from the sensing data, and then the monitoring device can continue the execution of steps S602 and S603 based on sensing data corresponding to the fit line(s) with positive changing rate.

In an implementation, in case the physical parameter is the pressure of the gas flow, step S601 can include: for the plurality of fit lines, determining whether changing rates of at least one pair of fit lines among the plurality of fit lines are all negative; if the changing rates of the at least one pair of fit lines among the plurality of fit lines are all negative, it is determined that breath(s) corresponding to the at least one pair of fit lines is real breath(s), and then the monitoring device can continue the execution of steps S602 and S603 based on the sensing data (the sensing data corresponding to the at least one pair of fit lines); if the changing rates of the at least one pair of fit lines among the plurality of fit lines are not all negative (i.e., at least one changing rate of at least one fit line is positive), it is determined that a breath corresponding to the fit line(s) with positive changing rate is not a real breath, sensing data corresponding to the fit line(s) with a positive changing rate is deleted from the sensing data, and then the monitoring device can continue the execution of steps S602 and S603 based on sensing data corresponding to the fit line(s) with negative changing rate.

In an implementation, in the case the physical parameter includes the flow rate and the pressure of the gas flow, step S502, step S603, and step S702 can include the following steps: determining whether the difference between a first physiological parameter obtained based on the flow rate of the gas flow and a first physiological parameter obtained based on the pressure of the gas flow is smaller than or equal to a preset value; if the difference between a first physiological parameter obtained based on the flow rate of the gas flow and a first physiological parameter obtained based on the pressure of the gas flow is smaller than or equal to a preset value, determining a first physiological parameter obtained based on the flow rate of the gas flow, a first physiological parameter obtained based on the pressure of the gas flow, or the average between a first physiological parameter obtained based on the flow rate of the gas flow and a first physiological parameter obtained based on the pressure of the gas flow, as the first physiological parameter; if the difference between a first physiological parameter obtained based on the flow rate of the gas flow and a first physiological parameter obtained based on the pressure of the gas flow is greater than the preset value, discarding the first physiological parameters obtained based on the flow rate and the pressure of the gas flow.

The above implementations are exemplary solutions for determining the first physiological parameter of the first user by the physical parameter of the gas flow at the side of the monitoring device, where the gas flow is delivered from the side of the monitoring device to the first user. In addition to the sensing data, other kinds of data can also be adopted for realizing the determination of the first physiological parameter. In the following, solutions for assessing breathing rate using temperature data or heating element data collected by a sensor positioned near the first user (i.e., at the side of the first user) will be illustrated. Through the solutions mentioned afore, the accuracy of the monitoring for the first physiological can be further improved. On the basis of providing the at least one sensing device within the monitoring device to determine the first physiological parameter, the solutions for determining the first physiological parameter based on the data collected by a sensor positioned near the first user can further improve the monitoring accuracy of the first physiological parameter monitored by the at least one sensing device within the monitoring device. In this way, the first physiological parameter monitored at the monitoring device side and the first physiological parameter monitored at the first user side can be compared for verification purpose.

In an implementation, the monitoring method can further include the following steps:
step S801, the monitoring device obtains temperature data measured by at least one temperature sensor, where the temperature data characterizes a temperature of the gas flow at a side of the first user, and a fluctuation of the temperature reflects an inhalation or exhalation of the first user; and
step S802, the monitoring device determines the first physiological parameter of the first user based on the temperature data.

In this implementation, the monitoring can obtain the temperature data measured by the at least one temperature sensor, herein the number of the at least one temperature sensor is not limited in the present disclosure. The breathing circuit is typically configured with a heating element (e.g., heating wire) to heat the gas flow. At least one temperature sensor can be provided at the very end of the breathing circuit towards the side of the first user, to control the heating to provide the gas flow with a set temperature to the first user. When the first user inhales, the temperature will be near the set temperature (e.g., the normal body temperature of a person, about 37° C.) and when the first user exhales, the temperature changes according to the exhalation temperature of the first user. These variations in the temperature can be captured from the temperature sensor reading to maintain the set temperature. That is, the temperature data measured by the at least one temperature sensor characterizes the temperature of the gas flow at the side of the first user, and the fluctuation of the temperature reflects the inhalation or exhalation of the first user so that the monitoring device can determine the first physiological parameter of the first user related to breath based on the temperature data.

In another implementation, in addition to the variations in the temperature measured by the at least one temperature sensor, the changes in control effort (heating power supply) of the heating element can be used to determine the first physiological parameter of the first user related to breath. In this implementation, the monitoring method can further include the following steps:
step S801, the monitoring device obtains a power change of a heating element, where the heating element is configured to heat the gas flow; and
step S802, the monitoring device determines the first physiological parameter of the first user based on the power change.

Since the heating power supply will be correspondingly adjusted when the temperature changes, the power change of the heating element can be also used to determine the first physiological parameter of the first user. For example, as mentioned above, when the first user inhales, the temperature of the gas flow will be near the set temperature (e.g., about 37° C.), in this case, the amplitude for adjusting the temperature of the heating element is relatively smaller, and the power change is small; when the first user exhales, the temperature of the gas flow will decrease, the amplitude for adjusting the temperature of the heating element is greater, and the power change is relatively greater. Therefore, the power change can reflect the breathing cycle, for example, the breathing cycle can be calculated by calculating the number of points with relatively larger power change in a predefined time length in a similar way as the solution of determining the first physiological parameter of the first user based on the peaks of the sensing data mentioned above.

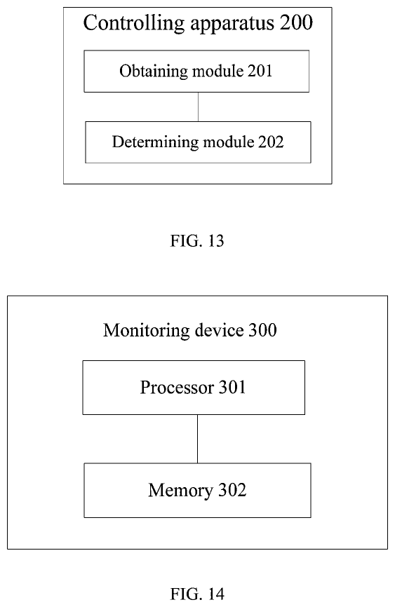

The present disclosure further provides a monitoring apparatus that can be configured to perform the monitoring method mentioned above. Since the principles of the method and the apparatus to solve the problem are similar, the definition and the explanation of the same terminologies used in the description of the monitoring apparatus can be referred to that in the description of the monitoring method, and the repetitive details will not be repeated. FIG. 13 is a schematic structural diagram of a monitoring apparatus according to an embodiment of the present disclosure. As shown in FIG. 13, the monitoring apparatus 200 can include:

an obtaining module 201, configured to obtain sensing data measured by at least one sensing device, where the at least one sensing device is provided within the monitoring apparatus 200, and the sensing data characterizes a physical parameter of a gas flow at a side of the monitoring apparatus 200, where the gas flow is delivered from the side of the monitoring apparatus 200 to a first user; and a determining module 202, configured to determine a first physiological parameter of the first user based on the sensing data.

The obtaining module 201 is communicatively connected to the at least one sensing device (not shown in FIG. 13) and the determining module 202, so as to obtain the sensing data measured by the at least one sensing device and exchange data with the determining module 202.

In an implementation, the at least one sensing device can include at least one flow sensor, and the physical parameter includes a flow rate of the gas flow at the side of the monitoring apparatus 200. In an implementation, the at least one sensing device can include at least one pressure sensor, and the physical parameter includes a pressure of the gas flow at the side of the monitoring apparatus 200. In an implementation, the at least one sensing device can include the at least one flow sensor and the at least one pressure sensor, and the physical parameter includes the flow rate and the pressure of the gas flow at the side of the monitoring apparatus 200.

In an implementation, the monitoring apparatus 200 can be applied to a high-flow device (e.g., integrated into the high-flow device), or the monitoring apparatus 200 can be a high-flow device.

In an implementation, the determining module 202 can be further configured to:
determine the first physiological parameter of the first user based on peaks of the sensing data or changing rates derived from the sensing data in a time domain or a maximum frequency component of the sensing data in a frequency domain.

In an implementation, the determining module 202 can be further configured to:
apply Fourier transform to the sensing data to obtain the maximum frequency component of the sensing data; and
determine a frequency corresponding to the maximum frequency component as the first physiological parameter.

In an implementation, the determining module 202 can be further configured to:
perform fitting on the sensing data to obtain a plurality of fit lines, where changing rates of the plurality of fit lines are all negative or positive;
calculate a time interval between at least one pair of fit lines among the plurality of fit lines; and determine the first physiological parameter according to the time interval.

In an implementation, the determining module 202 can be further configured to:
calculate a number of peaks of the sensing data over a predefined time length; and obtain the first physiological parameter based on the number of peaks and the predefined time length.

In an implementation, the obtaining module 201 can be further configured to:
obtain temperature data measured by at least one temperature sensor, where the temperature data characterizes a temperature of the gas flow at a side of the first user, and a fluctuation of the temperature reflects an inhalation or exhalation of the first user; and
the determining module 202 can be further configured to:
determine the first physiological parameter of the first user based on the temperature data.

In an implementation, the obtaining module 201 can be further configured to:
obtain a power change of a heating element, where the heating element is configured to heat the gas flow;
determine the first physiological parameter of the first user based on the power change.

In an implementation, the first physiological parameter can be a breathing rate.

The present disclosure further provides a monitoring device that can be configured to perform the monitoring method mentioned above. Since the principles of the method and the device to solve the problem are similar, the definition and the explanation of the same terminologies used in the description of the monitoring device can be referred to that in the description of the monitoring method, and the repetitive details will not be repeated. FIG. 14 is a schematic structural diagram of a monitoring device according to an embodiment of the present disclosure. As shown in FIG. 14, the monitoring device 300 can include:

a processor 301 and a memory 302 stored with instructions, where the processor 301 is configured to call and run the instructions stored in the memory 302 to execute operations of:
obtaining sensing data measured by at least one sensing device, where the at least one sensing device is provided within the monitoring device and the sensing data characterizes a physical parameter of a gas flow at a side of the monitoring device, where the gas flow is delivered from the side of the monitoring device to a first user; and
determining a first physiological parameter of the first user based on the sensing data.

In an implementation, the at least one sensing device (not shown in FIG. 14) can include at least one flow sensor, and the physical parameter includes a flow rate of the gas flow at the side of the monitoring device 300. In an implementation, the at least one sensing device can include at least one pressure sensor, and the physical parameter includes a pressure of the gas flow at the side of the monitoring device 300. In an implementation, the at least one sensing device can include the at least one flow sensor and the at least one pressure sensor, and the physical parameter includes the flow rate and the pressure of the gas flow at the side of the monitoring device 300.

In an implementation, the monitoring device 300 can be applied to a high-flow device (e.g., integrated into the high-flow device), or the monitoring device 300 can be a high-flow device.

In an implementation, the processor 301 is further configured to call and run the instructions stored in the memory to execute operations of:

determining the first physiological parameter of the first user based on peaks of the sensing data or changing rates derived from the sensing data in a time domain or a maximum frequency component of the sensing data in a frequency domain.

In an implementation, the processor 301 is further configured to call and run the instructions stored in the memory to execute operations of:
applying Fourier transform to the sensing data to obtain the maximum frequency component of the sensing data; and
determining a frequency corresponding to the maximum frequency component as the first physiological parameter.

In an implementation, the processor 301 is further configured to call and run the instructions stored in the memory to execute operations of:
performing fitting on the sensing data to obtain a plurality of fit lines, where changing rates of the plurality of fit lines are all negative or positive;
calculating a time interval between at least one pair of fit lines among the plurality of fit lines; and
determining the first physiological parameter according to the time interval.

In an implementation, the processor 301 is further configured to call and run the instructions stored in the memory to execute operations of:
calculating a number of peaks of the sensing data over a predefined time length; and
obtaining the first physiological parameter based on the number of peaks and the predefined time length.

In an implementation, the processor 301 is further configured to call and run the instructions stored in the memory to execute operations of:
obtaining temperature data measured by at least one temperature sensor, where the temperature data characterizes a temperature of the gas flow at a side of the first user, and a fluctuation of the temperature reflects an inhalation or exhalation of the first user; and determining the first physiological parameter of the first user based on the temperature data.

In an implementation, the processor 301 is further configured to call and run the instructions stored in the memory to execute operations of:
obtaining a power change of a heating element, where the heating element is configured to heat the gas flow;
determining the first physiological parameter of the first user based on the power change.

In an implementation, the first physiological parameter can be a breathing rate.

The present disclosure further provides a non-transitory computer-readable storage medium, which stores therein computer-executable instructions which, when being executed by one or more processors, implement the monitoring method according to embodiments of the present disclosure.

The present disclosure further provides a computer program, when the computer program is executed by one or more processors, implements the monitoring method according to embodiments of the present disclosure.

The present disclosure further provides a computer program product, which stores thereon computer executable instructions which, when being executed by one or more processors, implements the monitoring method according to embodiments of the present disclosure.

In one or more examples, the functions described may be implemented in hardware, software, firmware, or any combination thereof. For example, the functions may be implemented by one or more processors, such as one or more application-specific integrated circuits (ASICs), field programmable logic arrays (FPGAs), or other equivalent integrated or discrete logic circuitry. Accordingly, the term “processor,” as used herein may refer to any of the foregoing structures or any other structure suitable for the implementation of the techniques described herein. In addition, the techniques could be fully implemented in one or more circuits or logic elements.

Of course, the devices and components shown in the drawings may include further elements that are not shown in the drawings. The functions of the foreign object monitoring apparatus described in the specification can be realized by a circuit, which includes a subcircuit or a combination of a plurality of subcircuits, that is, the modules (the obtaining module and the determining module) described in the specification can be implemented as a subcircuit or a combination of a plurality of subcircuits.

The foregoing detailed description has been presented for purposes of illustration and description. It is not intended to be exhaustive or to limit the subject matter claimed herein to the precise form(s) disclosed. Many modifications and variations are possible in light of the above teachings. The described embodiments were chosen in order to best explain the principles of the disclosed technology and its practical application to thereby enable others skilled in the art to best utilize the technology in various embodiments and with various modifications as are suited to the particular use contemplated. Those embodiments with various modifications are within the range and scope of the following claims.

What is claimed is:

1. A method, wherein the method is applied to a monitoring device, and the method comprises:
obtaining sensing data measured by at least one sensing device, wherein the at least one sensing device is provided within the monitoring device and the sensing data characterizes a physical parameter of a gas flow at a side of the monitoring device, wherein the gas flow is delivered from the side of the monitoring device to a first user; and
determining a first physiological parameter of the first user based on the sensing data.

2. The method according to claim 1, wherein the at least one sensing device comprises at least one flow sensor, and the physical parameter comprises a flow rate of the gas flow at the side of the monitoring device.

3. The method according to claim 1, wherein the at least one sensing device comprises at least one pressure sensor, and the physical parameter comprises a pressure of the gas flow at the side of the monitoring device.

4. The method according to claim 1, wherein determining the first physiological parameter of the first user based on the sensing data comprises:
determining the first physiological parameter of the first user based on peaks of the sensing data or changing rates derived from the sensing data in a time domain or a maximum frequency component of the sensing data in a frequency domain.

5. The method according to claim 4, wherein determining the first physiological parameter of the first user based on the maximum frequency component of the sensing data in the frequency domain comprises:
applying Fourier transform to the sensing data to obtain the maximum frequency component of the sensing data; and
determining a frequency corresponding to the maximum frequency component as the first physiological parameter.

6. The method according to claim 4, wherein determining the first physiological parameter of the first user based on the changing rates derived from the sensing data in the time domain comprises:
performing fitting on the sensing data to obtain a plurality of fit lines, wherein changing rates of the plurality of fit lines are all negative or positive;
calculating a time interval between at least one pair of fit lines among the plurality of fit lines; and
determining the first physiological parameter according to the time interval.

7. The method according to claim 4, wherein determining the first physiological parameter of the first user based on the peaks of the sensing data in the time domain comprises:
calculating a number of peaks of the sensing data over a predefined time length; and
obtaining the first physiological parameter based on the number of peaks and the predefined time length.

8. The method according to claim 1, further comprising:
obtaining temperature data measured by at least one temperature sensor, wherein the temperature data characterizes a temperature of the gas flow at a side of the first user, and a fluctuation of the temperature reflects an inhalation or exhalation of the first user; and
determining the first physiological parameter of the first user based on the temperature data.

9. The method according to claim 1, further comprising:
obtaining a power change of a heating element, wherein the heating element is configured to heat the gas flow;
determining the first physiological parameter of the first user based on the power change.

10. The method according to claim 1, wherein the first physiological parameter is a breathing rate.

11. A monitoring device, comprising:
a memory stored with instructions and a processor, wherein the processor is configured to call and run the instructions stored in the memory to execute operations of:
obtaining sensing data measured by at least one sensing device, wherein the at least one sensing device is provided within the monitoring device and the sensing data characterizes a physical parameter of a gas flow at a side of the monitoring device, wherein the gas flow is delivered from the side of the monitoring device to a first user; and
determining a first physiological parameter of the first user based on the sensing data.

12. The monitoring device according to claim 11, wherein the at least one sensing device comprises at least one flow sensor, and the physical parameter comprises a flow rate of the gas flow at the side of the monitoring device.

13. The monitoring device according to claim 11, wherein the at least one sensing device comprises at least one pressure sensor, and the physical parameter comprises a pressure of the gas flow at the side of the monitoring device.

14. The monitoring device according to claim 11, wherein the processor is further configured to call and run the instructions stored in the memory to execute operations of:
determining the first physiological parameter of the first user based on peaks of the sensing data or changing rates derived from the sensing data in a time domain or a maximum frequency component of the sensing data in a frequency domain.

15. The monitoring device according to claim 14, wherein the processor is further configured to call and run the instructions stored in the memory to execute operations of:
applying Fourier transform to the sensing data to obtain the maximum frequency component of the sensing data; and
determining a frequency corresponding to the maximum frequency component as the first physiological parameter.

16. The monitoring device according to claim 14, wherein the processor is further configured to call and run the instructions stored in the memory to execute operations of:
performing fitting on the sensing data to obtain a plurality of fit lines, wherein changing rates of the plurality of fit lines are all negative or positive;
calculating a time interval between at least one pair of fit lines among the plurality of fit lines; and
determining the first physiological parameter according to the time interval.

17. The monitoring device according to claim 14, wherein the processor is configured to call and run the instructions stored in the memory to execute operations of:
calculating the number of peaks of the sensing data over a predefined time length; and
obtaining the first physiological parameter based on the number of peaks and the predefined time length.

18. The monitoring device according to claim 11, wherein the processor is configured to call and run the instructions stored in the memory to execute operations of:
obtaining temperature data measured by at least one temperature sensor, wherein the temperature data characterizes a temperature of the gas flow at a side of the first user, and a fluctuation of the temperature reflects an inhalation or exhalation of the first user; and
determining the first physiological parameter of the first user based on the temperature data.

19. The monitoring device according to claim 11, wherein the processor is configured to call and run the instructions stored in the memory to execute operations of:
obtaining a power change of a heating element, wherein the heating element is configured to heat the gas flow;
determining the first physiological parameter of the first user based on the power change.

20. The monitoring device according to claim 11, wherein the first physiological parameter of the first user is breathing rate.
